(19) United States
(12) Patent Application Publication
(10) Pub. No.: US 2020/0107855 A1
(43) Pub. Date: Apr. 9, 2020

(54) DEVICES AND METHODS FOR GENERATING ORBITAL MOTION IN DRIVE SHAFTS FOR ROTATIONAL MEDICAL DEVICES

(71) Applicant: Cardiovascular Systems, Inc., St. Paul, MN (US)

(72) Inventors: Joseph P. Higgins, Minnetonka, MN (US); Kayla J. Eichers, Minneapolis, MN (US)

(21) Appl. No.: 16/594,834

(22) Filed: Oct. 7, 2019

Related U.S. Application Data
(60) Provisional application No. 62/742,658, filed on Oct. 8, 2018.

(51) Int. Cl. A61B 17/3207

(52) U.S. Cl. CPC A61B 17/320758; A61B 2017/320004

(57) ABSTRACT

Devices, methods and systems are described that enable achieving a working diameter during high-speed rotation that is greater than a resting diameter. The various embodiments comprise structural modifications to rotational drive shafts that result in a radial shift of the center of mass of an affected portion of the drive shaft away from the rotational axis of the drive shaft. As a result, high-speed rotation of the drive shaft induces orbital motion. Certain aspects include more dense plugs inserted into or integrated into one or more spaced apart locations in one or more wire turns or filars along the drive shaft. One or more filars may comprise a denser portion than other filars. A flexible strip of preferably semi-circular cross-sectional shape may be affixed to the interior of the drive shaft within the lumen to add mass and affect the location of the center of mass.

### CROSS-REFERENCE TO RELATED APPLICATIONS

This application claims the benefit of U.S. Provisional Application No. 62/742,658, filed Oct. 8, 2018 and entitled ORBITAL MOTION WITH PREFORMED ROTATIONAL DRIVE SHAFT FOR ROTATIONAL MEDICAL DEVICES, the entirety of which is hereby incorporated by reference.

### STATEMENT REGARDING FEDERALLY SPONSORED RESEARCH OR DEVELOPMENT

Not Applicable

### BACKGROUND OF THE INVENTION

### Field of the Invention

The invention relates to drive shafts used in rotational medical devices including but not limited to orbital atherectomy devices and systems.

### Description of the Related Art

Rotational medical devices require a drive shaft that is rotated at high rotational speeds. For rotational atherectomy devices, it is known that adding an abrasive element to the drive shaft, wherein the abrasive element has a center of mass radially offset from the longitudinal axis of the drive shaft will achieve orbital motion during high-speed rotation. One of the characteristics of orbital motion is a working diameter achieved by the abrasive element during high-speed rotation that is greater than a resting diameter of the abrasive element. In these known systems, the abrasive element having a radially offset center of mass is referred to as “eccentric.” This eccentricity in terms of the radially offset center of mass may be achieved by a geometric asymmetry of the abrasive element, an asymmetric mounting of the abrasive element to the drive shaft and/or moving the center of mass of a symmetric abrasive element by, e.g., inserting a high-density plug of material into the abrasive element and/or removing some material from the abrasive element.

FIG. 1 illustrates a prior art device 100 comprising drive shaft 20 that is symmetric along its length. A symmetric and concentric burr 12 is mounted at the distal end of the drive shaft 20, wherein the burr 12 comprises a center of mass C that is located on the rotational axis A of the drive shaft 20. Drive shaft is translated within lumen of a catheter 13 and is connected at a proximal end to a prime mover located within a handle 10. Guide wire 15 is shown translated through the lumen of drive shaft 20 and through a lumen defined by burr 12. In this case, no asymmetry or eccentricity is present and, unless perturbed by bumping into an asymmetric object such as a lesion, the drive shaft 20 will not achieve orbital rotational motion as a result. Thus, the resting diameter of the drive shaft and of the burr will effectively be the same as the unperturbed working diameters of the drive shaft and of the burr during high-speed rotation.

The art progressed to form an enlarged and abrasive coated portion of the drive shaft as shown in FIGS. 2 and 3, wherein the wire turns of the drive shaft have been stretched by a shaped mandrel as is known in the art. The enlarged portion of the drive shaft may be symmetric and concentric in which case the center of mass will be located on the drive shaft's rotational axis and no orbital motion will result. Alternatively, the enlarged portion of the drive shaft may be asymmetric and eccentric wherein the center of mass is radially spaced away from the rotational axis of the drive shaft. The latter eccentricity results in generation of orbital motion of the eccentric enlarged drive shaft portion wherein the working diameter traced out by the enlarged portion is greater than its resting diameter.

Thus, FIG. 2, similar to FIG. 1, also provides a handle 10, an elongated flexible drive shaft 20 and an elongated catheter 13 extending distally from handle 10. The enlarged diameter portion 28 is formed from the wire turns of the drive shaft 20. Drive shaft 20 is formed or constructed from helically coiled wire turns. As known in the art, the drive shaft 20 may comprise one layer of helically coiled wire or two layers of helically coiled wire turns or filars as they are known in the art. In some cases, the two-layered embodiment of wire turns may comprise oppositely wound coils and in other cases the wire turns or filars of the two layers may be wound in the same direction. All such embodiments are within the scope of the present invention.

FIG. 2 further provides a guide wire 15 and a fluid supply line 17 for introducing a cooling and/or lubrication solution. A pair of optic cables 25 may be provided to monitor speed of rotation and the handle may include a control knob 11 the advance and/or retract the drive shaft.

FIG. 3 illustrates one embodiment of the enlarged drive shaft portion 28 of FIG. 2 in cutaway perspective view. Here, the enlarged wire turns or filars 41 of the drive shaft 20 are visible as is the exemplary abrasive coating 24 adhered thereto. As noted, the center of mass of enlarged portion 28 may be on the rotational axis A and, therefore concentric and not adapted to generate orbital motion during high-speed rotation. Alternatively the center of mass of enlarged portion 28 may be radially spaced away from the rotational axis A of the drive shaft 20 and thereby adapted to achieve orbital motion during high-speed rotation.

FIG. 4 provides another alternative as known in the art wherein a crown 28A is mounted to the drive shaft 20. The crown 28A, as shown, is eccentric and/or eccentrically mounted to the drive shaft 28A to provide a center of mass C that is located radially away from the rotational axis A of the drive shaft 20. As with the eccentric embodiment discussed above in connection with FIGS. 3 and 4, the eccentric crown 28A will be urged into orbital motion during high-speed rotation of the drive shaft 20 wherein its working diameter traced out during rotation is greater than its resting diameter. The center of mass C location may be manipulated by modifying a number of elements as the skilled artisan will understand, including but not limited to the provision of a hollowed chamber 30 within the crown 28A. If present, the size and/or shaping of the hollowed chamber 30 may be changed to manipulate the center of mass C location.

A final exemplary prior art embodiment is illustrated in FIG. 5 wherein the desired eccentricity to generate orbital motion is provided by a pre-curved section 28B of the drive shaft 20. This arrangement radially spaces the center of mass C of the pre-curved section, and the accompanying abrasive section 24 which may be an abrasive coating as shown or a burr or crown attached thereto, away from the rotational axis A of the drive shaft 20. Consequently, high-speed rotation of this drive shaft 20 will result in a tracing of the abrasive section 24 having a working diameter that is great than its resting diameter.

FIG. 6 illustrates a cross-sectional view of the wire turns or filars 41 of a prior art drive shaft 20 and the defined lumen L therethrough. Generally, this prior art device is symmetric and concentric about the rotational axis and, therefore, the center of mass C at any point along the drive shaft's length will be located on the rotational axis A of the drive shaft and, without more, orbital motion will not be induced or achieved.

It would be desirable to provide a mechanism for achieving orbital motion by means that do not involve or require an eccentric abrasive element or other prior art means discussed above. Accordingly, in such a system, the abrasive element need not be present in the case where the drive shaft 20 and wire turns or filars 41 is/are coated with abrasive, need not be “eccentric” if an abrasive element such as a crown or burr is present and may in fact be concentric when an abrasive element is present.

Various embodiments of the present invention address these, inter alia, issues.

Moreover, we provide disclosure of the following patents and applications, each of which are assigned to Cardiovascular Systems, Inc., and incorporated herein in their entirety, each of which may comprise systems, methods and/or devices that may be used with various embodiments of the presently disclosed subject matter:

U.S. Pat. No. 9,468,457, “ATHERECTOMY DEVICE WITH ECCENTRIC CROWN”;

U.S. Pat. No. 9,439,674, “ROTATIONAL ATHERECTOMY DEVICE WITH EXCHANGEABLE DRIVE SHAFT AND MESHING GEARS”;

U.S. Pat. No. 9,220,529, “ROTATIONAL ATHERECTOMY DEVICE WITH ELECTRIC MOTOR”;

U.S. Pat. No. 9,119,661, “ROTATIONAL ATHERECTOMY DEVICE WITH ELECTRIC MOTOR”;

U.S. Pat. No. 9,119,660, “ROTATIONAL ATHERECTOMY DEVICE WITH ELECTRIC MOTOR”;

U.S. Pat. No. 9,078,692, “ROTATIONAL ATHERECTOMY SYSTEM”;

U.S. Pat. No. 6,295,712, “ROTATIONAL ATHERECTOMY DEVICE”;

U.S. Pat. No. 6,494,890, “ECCENTRIC ROTATIONAL ATHERECTOMY DEVICE”;

U.S. Pat. No. 6,132,444, “ECCENTRIC DRIVE SHAFT FOR ATHERECTOMY DEVICE AND METHOD FOR MANUFACTURE”;

U.S. Pat. No. 6,638,288, “ECCENTRIC DRIVE SHAFT FOR ATHERECTOMY DEVICE AND METHOD FOR MANUFACTURE”;

U.S. Pat. No. 5,314,438, “ABRASIVE DRIVE SHAFT DEVICE FOR ROTATIONAL ATHERECTOMY”;

U.S. Pat. No. 6,217,595, “ROTATIONAL ATHERECTOMY DEVICE”;

U.S. Pat. No. 5,554,163, “ATHERECTOMY DEVICE”;

U.S. Pat. No. 7,507,245, “ROTATIONAL ANGIOPLASTY DEVICE WITH ABRASIVE CROWN”;

U.S. Pat. No. 6,129,734, “ROTATIONAL ATHERECTOMY DEVICE WITH RADIALLY EXPANDABLE PRIME MOVER COUPLING”;

U.S. patent application Ser. No. 11/761,128, “ECCENTRIC ABRADING HEAD FOR HIGH-SPEED ROTATIONAL ATHERECTOMY DEVICES”;

U.S. patent application Ser. No. 11/767,725, “SYSTEM, APPARATUS AND METHOD FOR OPENING AN OCCLUDED LESION”;

U.S. patent application Ser. No. 12/130,083, “ECCENTRIC ABRADING ELEMENT FOR HIGH-SPEED ROTATIONAL ATHERECTOMY DEVICES”;

U.S. patent application Ser. No. 12/363,914, “MULTI-MATERIAL ABRADING HEAD FOR ATHERECTOMY DEVICES HAVING LATERALLY DISPLACED CENTER OF MASS”;

U.S. patent application Ser. No. 12/578,222, “ROTATIONAL ATHERECTOMY DEVICE WITH PRE-CURVED DRIVE SHAFT”;

U.S. patent application Ser. No. 12/130,024, “ECCENTRIC ABRADING AND CUTTING HEAD FOR HIGH-SPEED ROTATIONAL ATHERECTOMY DEVICES”;

U.S. patent application Ser. No. 12/580,590, “ECCENTRIC ABRADING AND CUTTING HEAD FOR HIGH-SPEED ROTATIONAL ATHERECTOMY DEVICES”;

U.S. patent application Ser. No. 29/298,320, “ROTATIONAL ATHERECTOMY ABRASIVE CROWN”;

U.S. patent application Ser. No. 29/297,122, “ROTATIONAL ATHERECTOMY ABRASIVE CROWN”;

U.S. patent application Ser. No. 12/466,130, “BIDIRECTIONAL EXPANDABLE HEAD FOR ROTATIONAL ATHERECTOMY DEVICE”; and

U.S. patent application Ser. No. 12/388,703, “ROTATIONAL ATHERECTOMY SEGMENTED ABRADING HEAD AND METHOD TO IMPROVE ABRADING EFFICIENCY”.

### BRIEF SUMMARY OF THE INVENTION

Devices, methods and systems are described that enable achieving a working diameter during high-speed rotation that is greater than a resting diameter. The various embodiments comprise structural modifications to rotational drive shafts that result in a radial shift of the center of mass of an affected portion of the drive shaft away from the rotational axis of the drive shaft. As a result, high-speed rotation of the drive shaft induces orbital motion. Certain aspects include plugs inserted into or integrated into one or more spaced apart locations in one or more wire turns or filars along the drive shaft. One or more filars may comprise a denser portion than other filars. A flexible strip of preferably semi-circular cross-sectional shape may be affixed to the interior of the drive shaft within the lumen to add mass and affect the location of the center of mass.

The figures and the detailed description which follow more particularly exemplify these and other embodiments of the invention.

### BRIEF DESCRIPTION OF THE SEVERAL VIEWS OF THE DRAWINGS

FIG. 1 is a cross-sectional view of a prior art device.

FIG. 2 is a perspective view of a prior art device.

FIG. 3 is a perspective cutaway view of a prior art device.

FIG. 4 is a cross-sectional and cutaway view of a prior art device.

FIG. 5 is a cross-sectional and cutaway view of a prior art device.

FIG. 6 is a cross-sectional view of a prior art drive shaft with guidewire.

FIG. 7A is a cutaway view of one embodiment of the present invention.

FIG. 7B is an end view of the embodiment of FIG. 7A.

FIG. 7C is an end view of the unaffected portion of FIG. 7A.

FIG. 8A is a cutaway view of one embodiment of the present invention.

FIG. 8B is an end view of the embodiment of FIG. 8A.

FIG. 9A is a cutaway view of one embodiment of the present invention.

FIG. 9B is an end view of the embodiment of FIG. 9A.

FIG. 9C is a schematic end view of rotationally spaced centers of mass of one embodiment of the present invention.

FIG. 10A is an end view of one embodiment of the present invention.

FIG. 10B is a side cutaway view of the embodiment of FIG. 10A.

FIG. 11A illustrates the drag coefficient of an exemplary circular object.

FIG. 11B illustrates the drag coefficient (“C_{D}”) of an exemplary square object.

FIG. 11C illustrates the drag coefficient (“C_{D}”) of an exemplary tilted square or diamond shaped object.

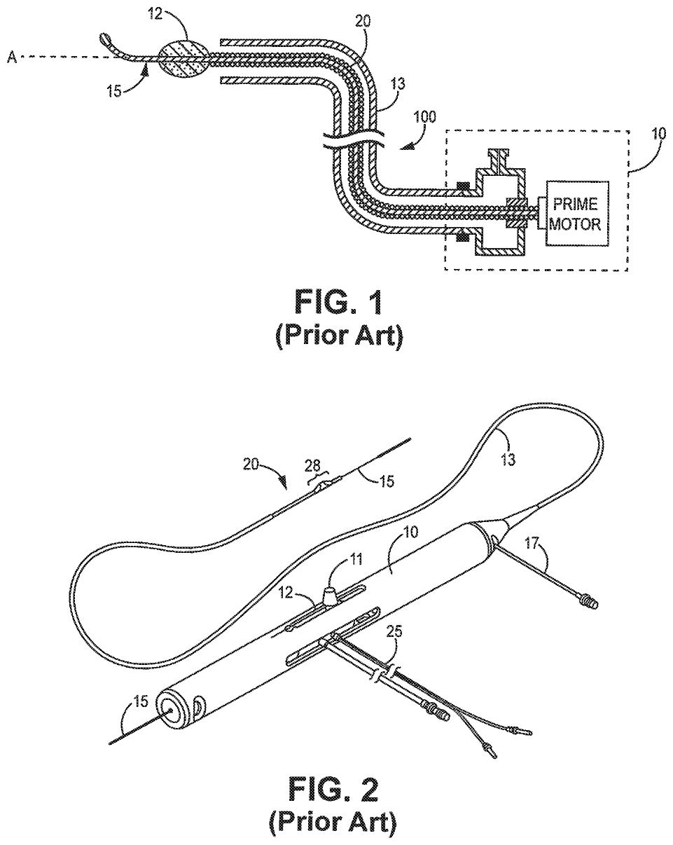

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Various embodiments of a rotational drive shaft for a rotational medical device such as a rotational atherectomy system are provided. Each embodiment generates orbital motion, derived from features integrated with the drive shaft not from an attached abrasive element.

Initially, it is to be understood that, as used herein and defined hereby, the word “eccentricity” and variants thereof refers to either (1) a difference in location between the geometric center of the drive shaft and the rotational axis of the drive shaft, or (2) a difference in location between the center of mass of the drive shaft and the rotational axis of the drive shaft.

Moreover, it is to be understood that, as used herein and defined hereby, the term “orbital motion” refers to the orbiting element, e.g., the drive shaft, achieving a working diameter that is larger than its resting diameter and wherein the orbital motion is induced by an eccentricity mounted on or in or along the drive shaft, in certain embodiments integrated in, along or on the wire turns or filars of the drive shaft. The resulting movement of the drive shaft during orbital motion may also be referred to as a standing wave of predictable, customizable length and shape.

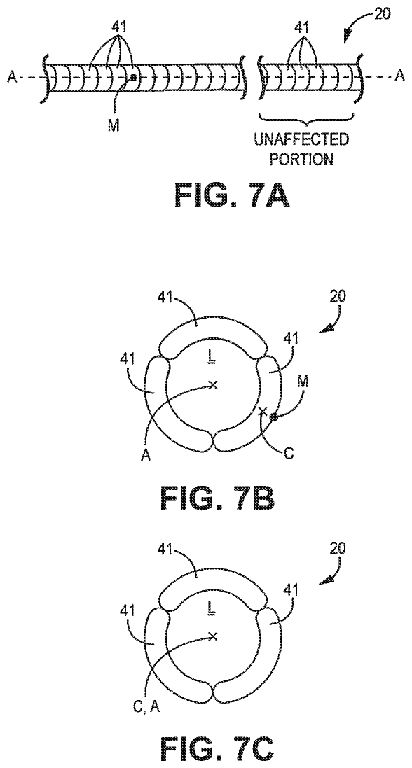

Turning to FIGS. 7A-9B, embodiments of one aspect of the present invention generally comprises at least one non-abrasive mass M incorporated within and/or along the wire turns or filars 41 of drive shaft 20 to move the center of mass of the affected drive shaft 20 section radially away from the axis of rotation of the drive shaft 20. In turn, orbital motion will be generated during high-speed rotation, wherein a working diameter achieved by the drive shaft 20 is greater than its resting diameter. At a point distal and/or proximal to the affected drive shaft section, the remaining drive shaft portions will be unaffected by mass(es) M and, therefore, will rotate generally with a working diameter roughly equal to the resting diameter of the drive shaft.

In addition, incorporating non-abrasive mass(es) M in or along the cross-section of the shaft may cause the shaft to wobble as it spins. This wobble, which differs from, and may occur concurrently with, the enlarged tracing achieved during orbital motion, will be sufficient, especially in smaller diameter arteries, to stir the fluid media to circulate around the artery which will in turn drive the atherectomy device to orbit around the artery or other vessel. Portions of the orbiting and/or wobbling drive shaft sections may comprise an abrasive coating.

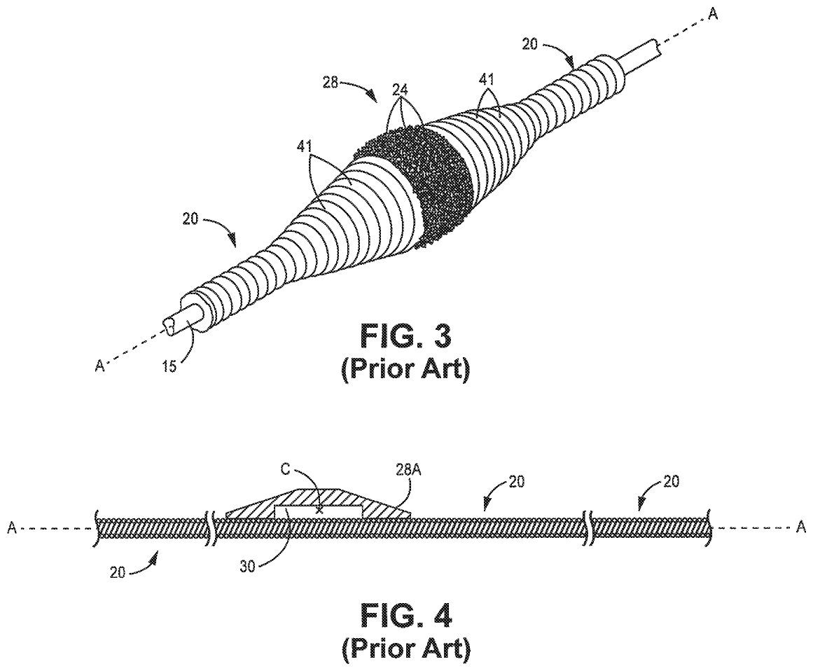

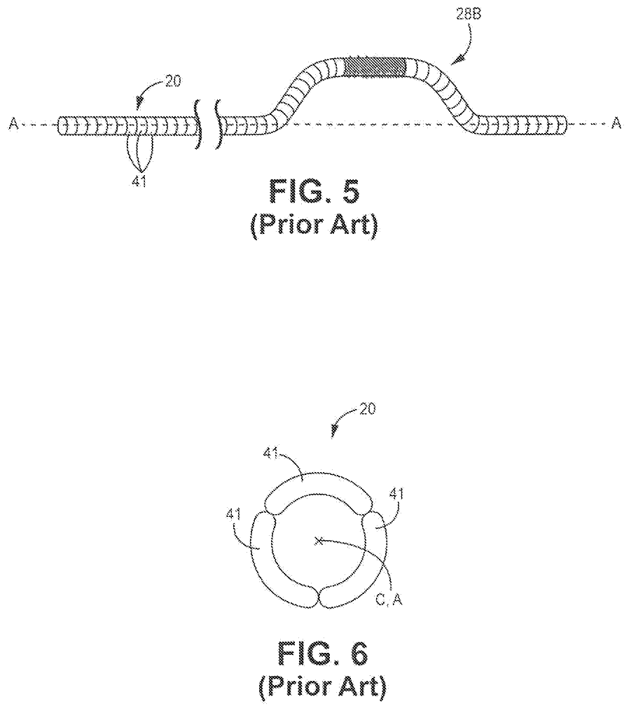

Further, because orbital motion and/or wobbling is induced by mass M in, on or along the drive shaft, a separate concentric abrasive element may be operatively connected to the orbiting and/or wobbling sections of the drive shaft. Without the mass M, the concentric abrasive element will not achieve orbital motion or wobble as it spins. However, mass M now enables inducement of the concentric abrasive element as discussed in connection with FIGS. 2 and 3 to achieve orbital motion. As mentioned, this structure has advantages over eccentric abrasive elements including but not limited to easier crossing of tortuous vasculature in certain cases, particularly very small diameter vessels.

Thus, an advantage of the above embodiments is that an eccentric crown is not required in order to sustain luminal orbit or orbital motion. This means it is particularly well-suited to deliver therapy in very small arteries or where the therapy access site requires a small introducer.

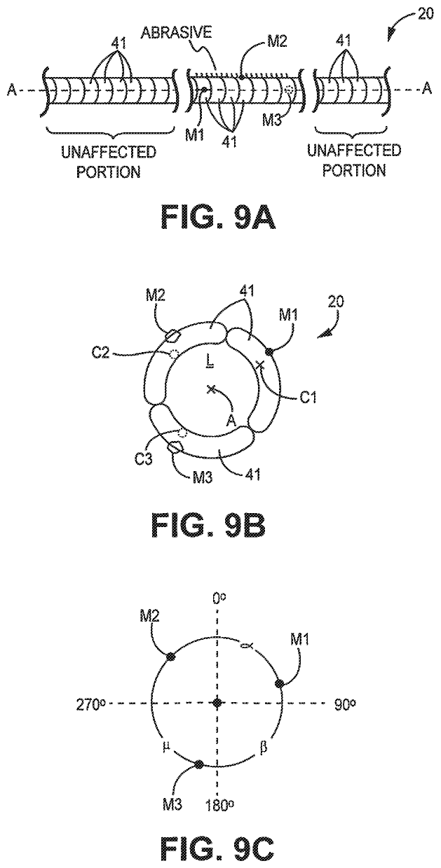

FIGS. 7A-7C illustrate an exemplary embodiment comprising one mass M attached to a wire turn or filar 41 at a single location. As discussed mass M may be affixed on or along a wire turn or filar 41. This means mass M may be mounted or affixed on the exterior or the interior (i.e., within the drive shaft lumen) of the wire turn or filar 41. Alternatively, mass M may be integrated within a wire turn or filar 41 at a single location. As shown in FIG. 9A, some or all of the wire turns or filars 41 affected by the mass M, i.e., the section of drive shaft 20 that achieves orbital motion, may be coated with an abrasive. FIG. 7B is an end view of the drive shaft 20 of FIG. 7A, illustrating the center of mass C location, as a result of the mass M affixation or integration position, radially offset from the drive shaft's rotational axis A. FIG. 7C shows an unaffected section or portion of the drive shaft 20 that is unaffected by the presence of mass M, i.e., wherein the center of mass C is located along the center of axis A and therefore does not achieve orbital motion or wobble. Thus, FIG. 7C illustrates the axis of rotation A of drive shaft 20 and the center of mass C as coextensive, i.e., the center of mass C is on the axis of rotation across the length of the unaffected portion. FIG. 7C's unaffected portion may be located proximal and/or distal to the affected section of FIG. 7B.

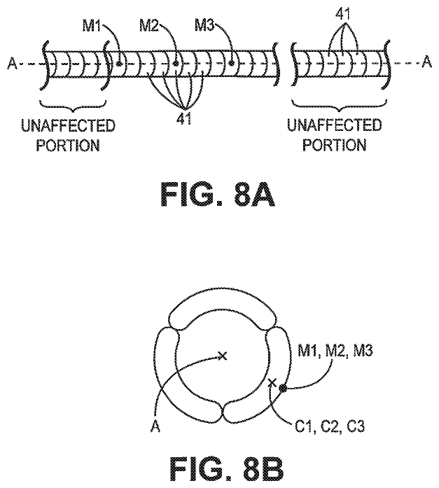

FIG. 8A is similar to FIG. 7A except that more than one mass M is affixed on, in or along the drive shaft wire turns or filars 41. In the illustrated case, 3 such masses M1, M2 and M3 are provided in spaced apart relationship to each other and generally are along the same longitudinal plane and with no rotational angles therebetween when viewed down the rotational axis A. Unaffected portion is the same as described in FIG. 7A. FIG. 8B is an end view showing that the resultant positions of the centers of mass C1, C2, C3 as radially offset from the drive shaft's nominal and central rotational axis A and in substantial alignment longitudinally without rotational angle separating any of the locations as a result of the alignment of masses M1, M2 and M3.

FIG. 9A illustrates a variation of the above embodiments, wherein the masses M1, M2, M3 are affixed on or along or integrated within drive shaft's wire turns or filars 41 in spaced apart longitudinal locations and spaced rotationally apart by rotational angles α, β, and μ separating the locations of M1 to M2 (α), M1 to M3 (β), and M2 to M3 (μ) when viewed down the rotational axis A. In addition, some or all of the wire turns or filars 41 affected by the masses M1-M3 may comprise an abrasive coating as shown. FIG. 9B is an end view showing the relative radial offset of each of the spaced apart masses M1-M3 and further illustrating the radial angling and/or spacing therebetween when viewed down the rotational axis A.

In all cases, mass(es) M may comprise an insert of higher density than the density of the wire turns or filars 41 into which mass(es) M is/are inserted or integrated; a smoothed very low profile node comprising a higher density than the density of the wire turns or filers 41.

In addition to the mass(es) M, concentric abrasive element(s) may be provided on one or more of the affected sections of drive shaft 20 to enable orbital motion of the concentric abrasive element(s).

While the offsetting mass(es) M may be sufficient to induce and sustain orbital motion as described above, one or more additional elements may also be placed along the shaft to encourage wobble and/or orbital motion and/or standing wave formation along affected portions of the drive shaft 20. These one or more elements may be concentric or eccentric, wherein concentric is defined to include geometric symmetry and/or a center of mass located at a geometric center of the element and wherein eccentric is defined to include geometric asymmetry and/or a center of mass located or spaced away from a geometric center of the element. These one or more elements could be abrasive or non-abrasive and may be generally located with the offsetting mass that induces orbital motion or may be spaced apart therefrom.

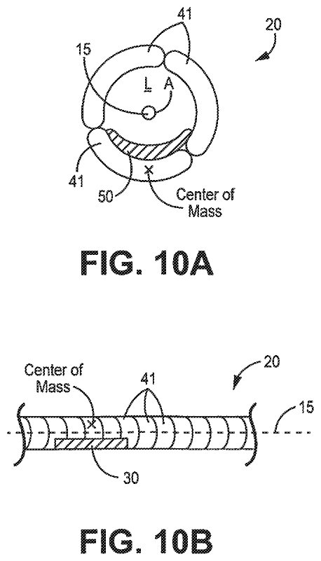

For example, a flexible strip 50 having a mass may be applied or adhered or affixed to one or more wire turns or filars 41 to create eccentricity in that region of the drive shaft. As shown in FIGS. 10A and 10B, the flexible strip 10 may be affixed on the interior of the drive shaft wire turns or filars 41, thus reducing the area of the lumen L defined therein at that location. As shown, the flexible strip 50 comprises a length and may comprise a semi-circular shape as shown in longitudinal cross-section to form a complementary surface to accommodate the circular guidewire 15 that is translated and/or rotated therealong. The flexible strip 50 may be used alone or in combination with the mass(es) M described above to generate orbital motion/standing wave of a size and shape and/or wobble of affected portions of the drive shaft 20.

As shown, the flexible strip 50 is semi-circular to induce the movement of the center of mass radially away from the axis of rotation A. Alternatively, the flexible strip 50 may comprise a circular insert surrounding or lining the interior of lumen L, but wherein a portion(s) of the flexible strip 50 are denser or more massive than the other portions to provide the desired mass eccentricity and resulting radially offset center of mass.

The standing wave induced on the drive shaft 20 by the above-described structure(s) is typically observed to have a nearly planar or spiral-shape. The mass(es) M and/or flexible strip comprise design parameters that, in turn, enables selection of the standing wave shape and/or length. For example:

Constant Circumferential Position;

Circumferential spiral with spin in same direction as spiraling or orbiting; and

Circumferential spiraling or orbiting with spin in the opposite direction as the spiraling or orbiting may all be achieved using embodiments of the present invention.

Another embodiment to induce wobbling or orbital motion/standing wave for drive shafts (20) comprises a multi-filar shaft 20 wherein the filars 41 are typically made of stainless steel. Here, one or more filars 41 may be replaced with a relatively higher density filars 41 than the exemplary stainless steel filars, e.g., tungsten, along at least a portion of the length of the drive shaft 20 move the center of mass C of that section of the drive shaft radially away from the axis of rotation A. Alternatively and/or in addition, at least some of at least a portion or length the remaining filars 41 may be replaced with a material of lower density than the remaining filars 41 along at least a portion of the length of the drive shaft 20.

It is important during an exemplary atherectomy or other tissue or material removal procedure involving a rotational drive shaft to remove the resultant material, particles and/or debris away from the abrasive region which may comprise a crown or burr or abrasive coating as described above, or a tissue cutter or macerator.

Thus, certain embodiments may take advantage of the winding direction of drive shaft's filars 41 to drive fluid toward or away from an abrasive crown or other abrasive section(s) located along the drive shaft 20. Depending on the winding direction of the driveshaft filars 41, its spin direction could be adjusted in such a way so that filars 41 “open” during spinning and drive fluid either forward, or backward. This is similar to “augering” and is a similar mechanism to a grain auger or an Archimedes screw. Similarly, driveshaft wire turns of filars 41 could be purposely stretched and heat set, or welded, to maintain openings between the filars 41 for this same purpose.

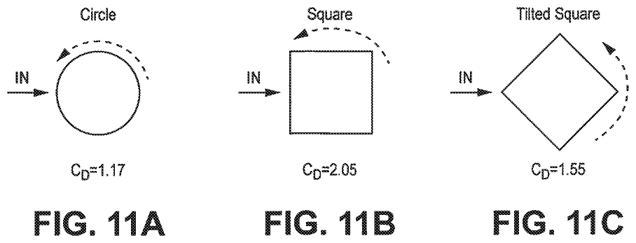

Alternatively, features may be placed on or affixed to the drive shaft 20 to function to drive fluid in a specific direction and typically in a direction away from the cutting or maceration or abrading region. Such features may comprise in some embodiments “vortex generators” V that would ultimately cause fluid to rotate in a specified direction which in turn could direct flow toward or away from the crown through the creation of turbulence and modification of pressure gradients and may comprise nodes that interrupt the otherwise relatively smooth longitudinal profile of the drive shaft. Such vortex generators V may be attached or affixed to the drive shaft. Alternatively, at least a portion of the drive shaft 20 may be shaped as a vortex generator V. As shown in FIGS. 11A-11C, drag coefficients of end views of a stationary circular cross-section drive shaft 20 and two different orientations of a square shaft are provided with inflowing fluid designated by the arrow in each case and approximating the flow of fluid during rotation of same (indicated by dashed arrows). There are many cross-sectional shapes or surface treatments which would increase the drag coefficient. The non-circular cross-sectional shape may comprise an ellipse, an oval, a square including a square with 90 degree sharp corners or smoothly radiused corners. Additional non-circular cross-sectional shapes will now become obvious to the skilled artisan and are within the scope of the present invention.

Accordingly, a drive shaft 20 with a non-circular longitudinal profile along at least a portion of the length of the drive shaft 20 may be implemented to increase the shaft's drag coefficient and maximize the resulting flow of fluid away from the cutting, maceration and/or abrading, with entrainment of the resultant material and/or debris.

This, a rotating drive shaft 20 with a non-circular longitudinal profile will increase the drag coefficient of the shaft which will:

1. more effectively stir the fluid to circulate and/or

2. more effectively be pushed by circulating fluid to orbit.

The descriptions of the embodiments and their applications as set forth herein should be construed as illustrative, and are not intended to limit the scope of the disclosure. Features of various embodiments may be combined with other embodiments and/or features thereof within the metes and bounds of the disclosure. Upon study of this disclosure, variations and modifications of the embodiments disclosed herein are possible, and practical alternatives to and equivalents of the various elements of the embodiments will be understood by and become apparent to those of ordinary skill in the art. Such variations and modifications of the embodiments disclosed herein may be made without departing from the scope and spirit of the invention. Therefore, all alternatives, variations, modifications, etc., as may become to one of ordinary skill in the art are considered as being within the metes and bounds of the instant disclosure.

What is claimed is:

1. A rotational medical device having a prime mover that is operationally rotationally connected to a drive shaft that is adapted to achieve a working diameter that is greater than its resting diameter during high-speed rotation, the drive shaft comprising:
a construction of more than one wire filar,
wherein one of the more than one filars comprises a material that is more dense than the remaining filars of the drive shaft.

2. The rotational medical device of claim 1, wherein material that is more dense than the remaining filars does not extend along the entire length of the filar.

3. The rotational medical device of claim 1, wherein at least a portion of the drive shaft is coated with abrasive material.

4. The rotational medical device of claim 1, further comprising a concentric abrasive element attached to the drive shaft and adapted to achieve a working diameter that is greater than its resting diameter during high-speed rotation.

5. A rotational medical device having a prime mover operatively rotationally connected with a drive shaft constructed of more than one filar and that is adapted to achieve a working diameter that is greater than its resting diameter during high-speed rotation, the drive shaft comprising:
a mass inserted or integrated into a location within a filar of the drive shaft.

6. The rotational medical device of claim 5, further comprising:
more than one mass inserted or integrated into spaced apart locations within one or more than one filar of the drive shaft.

7. The device of claim 6, wherein the spaced apart locations comprise longitudinal and/or rotational spacing.

8. The device of claim 6, wherein the spaced apart locations comprise longitudinal spacing.

9. The device of claim 8, wherein the spaced apart locations are aligned in the same longitudinal plane.

10. The device of claim 7, further comprising abrasive coating on an external surface of at least a portion of the drive shaft's filars.

11. The device of claim 7, further comprising a concentric abrasive element attached to the drive shaft and adapted to achieve a working diameter during high-speed rotation that is greater than its resting diameter.

12. A rotational medical device having a prime mover operatively rotationally connected with a drive shaft constructed of more than one filar and that is adapted to achieve a working diameter that is greater than its resting diameter during high-speed rotation, the rotational medical device comprising:
a flexible strip inserted into a portion of a lumen defined by the drive shaft and affixed to the inner wall of the drive shaft, wherein the flexible strip comprises a semi-circular longitudinal cross-sectional shape, a mass and a length.

13. The rotational medical device of claim 12, further comprising abrasive coating on at least an external surface of the drive shaft's filars.

14. The device of claim 12, further comprising a concentric abrasive element attached to the drive shaft and adapted to achieve a working diameter during high-speed rotation that is greater than its resting diameter.

15. The rotational medical device of claim 12, further comprising:
one or more masses inserted or integrated into spaced apart locations within one or more than one filar of the drive shaft.

16. The device of claim 15, wherein the spaced apart locations comprise longitudinal and/or rotational spacing.

17. The device of claim 15, wherein the spaced apart locations comprise longitudinal spacing.

18. The device of claim 17, wherein the spaced apart locations are aligned in the same longitudinal plane.

19. A rotational medical device comprising a prime mover in operational rotational connection with a flexible drive shaft having a length, the drive shaft comprising:
a non-circular radial cross-sectional profile extending at least a portion of the distance of the length of the drive shaft.

20. The rotational medical device of claim 19, wherein the non-circular radial cross-sectional profile comprises at least one of the group consisting of an ellipse, an oval, and a square.
